(19) United States
(12) Patent Application Publication
(10) Pub. No.: US 2012/0301928 A1
(43) Pub. Date: Nov. 29, 2012

(54) PREPROCESSING METHOD OF HYDROLYZING LIGNOCELLULOSIC BIOMASS AND METHODS OF MANUFACTURING SUGAR COMPOUND AND BIOETHANOL FROM BIOMASS PROCESSED USING THE PREPROCESSING METHOD

(75) Inventors: Hyeun Jong Bae, Gwangju (KR); Seung Gon Wi, Gwangju (KR); Han Ki Shin, Seongnam-si (KR)

(73) Assignee: INDUSTRY FOUNDATION OF CHONNAM NATIONAL UNIVERSITY, Gwangju (KR)

(21) Appl. No.: 13/574,403

(22) PCT Filed: Jan. 28, 2011
(86) PCT No.: PCT/KR2011/000622

§ 371 Date: Jul. 20, 2012

(30) Foreign Application Priority Data
Jan. 29, 2010 (KR) 10-2010-0008497

(51) Int. Cl. C12P 19/14; C08B 1/00; C12P 7/14

(52) U.S. Cl. 435/99; 435/162; 127/34

(57) ABSTRACT

The present invention relates to a method of pretreating lignocellulosic biomass prior to hydrolysis, and, more particularly, to a method of pretreating lignocellulosic biomass prior to hydrolysis using wet milling in combination with popping, a method of manufacturing a sugar compound from the biomass pretreated using the method, and a method of manufacturing bioethanol from the biomass pretreated using the method. According to the present invention, energy consumption can be reduced, lignocellulosic biomass can be easily pretreated in an environment-friendly manner without treating chemicals, and the efficiency of hydrolyzing lignocellulosic biomass can be remarkably improved.

### CROSS REFERENCE TO RELATED APPLICATIONS AND CLAIM OF PRIORITY

This patent application is a National Phase application under 35 U.S.C. §371 of International Application No. PCT/KR2011/000622, filed on Jan. 28, 2011, which claims priority to Korean Patent Application number 10-2010-0008497 filed Jan. 29, 2010, entire contents of which are incorporated herein by reference.

### BACKGROUND

1. Technical Field

The present invention relates to a method of pretreating lignocellulosic biomass prior to hydrolysis, and, more particularly, to a method of pretreating lignocellulosic biomass prior to hydrolysis using wet milling in combination with popping, a method of manufacturing a sugar compound from the biomass pretreated using the method, and a method of manufacturing bioethanol from the biomass pretreated using the method.

2. Related Art

Conventionally, sugar compounds are produced by culturing natural products such as plants or sea algae using microbes, and have been variously used in the fields of foods and medicine. Glucose is a major one of these sugar compounds. Glucose is used in many fermentation technologies as well as to meet energy demands, thus requiring various glucose supply sources.

Moreover, recently, in order to solve the problems of the greenhouse effect caused by global warming and the depletion of oil, methods of producing bio-energy using sugar compounds have attracted considerable attention. In this case, sugarcane juice or corn starch has been used as a carbohydrate source for producing bio-energy, that is, bioethanol.

However, raw materials for producing first-generation bioethanol (starch bioethanol) are encountering many problems, such as the competition with foods and cattle feed, the saturation of the region for growing the raw materials, and the like. Therefore, in order to solve these problems, research into second-generation bioethanol (cellulose ethanol) produced from lignocellulose extracted from woody and herbaceous plant is being done in the U.S.A, etc.

The reaction mechanism for producing fuel ethanol from pretreated lignocellulosic biomass largely includes the two processes of saccharification and fermentation. Saccharification is a process of converting cellulose into glucose using an enzyme. This process of converting cellulose into glucose includes a process of changing cellulose into cellobiose by adsorbing cellulase (enzyme) on the reaction surface of the cellulose and a process of converting the cellobiose into glucose using the enzyme reaction of β-glucosidase. Also, fermentation is the process of converting the glucose produced by the saccharification of cellulose into ethanol and carbon dioxide using a microbe such as yeast under an anaerobic condition.

As such, when lignocellulosic biomass is decomposed or converted, materials useful to human life, such as cellulose, pulp, glucose, xylose (xylan), lignin, ethanol, xylitol and the like, can be obtained in large quantities. In this case, the crystallinity and porosity of cellulose act as factors having a great influence on the biotechnological hydrolysis of lignocellulosic biomass, and the content of lignin or hemicellulose is an important factor, too.

In particular, in the biological hydrolysis of lignocellulosic biomass using an enzyme, the adsorption ability of an enzyme has a very close relationship with the surface area of cellulose. The hydrolysis rate of lignocellulosic biomass increases as the amount of adsorbed enzyme increases, and is determined by the amount of adsorbed enzyme and the crystallinity of cellulose.

Meanwhile, it is known that a pretreatment process is required to saccharify lignocellulosic biomass. For the pretreatment process to be effective, the amount of cellulose is increased, and the crystallinity of fine fiber is decreased, so that the adsorption rate of an enzyme per unit area of lignocellulosic biomass is increased, with the result that the reactivity of cellulose is increased, thereby increasing the hydrolyzability of an enzyme. The pretreatment process may be performed by various physical and chemical methods, such as steam explosion, alkali treatment, sulfur dioxide treatment, hydrogen peroxide treatment, supercritical ammonia treatment, weak acid extraction treatment, ammonia freezing explosion and the like. In actuality, the pretreatment process may also be performed by the combination of these methods.

In particular, steam explosion is disadvantageous in that the recovery rate of hemicellulose is low, and various kinds of non-recoverable byproducts, such as acetic acid (fermentation inhibitor), furfural and the like, are produced in large amounts. Further, when steam explosion is used as the pretreatment process of biomass, the saccharification efficiency of biomass is low. Therefore, a pretreatment process using a base, sulfur dioxide or hydrogen peroxide as the catalyst for decomposing a weak acid, organic solvent or liquid ammonia is used. However, this method is also disadvantageous in that expensive chemicals must be used, and an apparatus for recovering these chemicals is additionally required, thus increasing installation expenditures. For this reason, steam explosion has been barely put into practical use.

Meanwhile, steam explosion using ammonia, having lately attracted considerable attention as a pretreatment process, requires various kinds of devices, such as a preheating coil, a leaching reactor, a steam jacket, a temperature sensor, a control valve, a trap, a switching valve, a lignin discharge valve, an ammonia charge valve, an ammonia reheater, a lignin separator, an ammonia collector, a conductivity gauge, a conductivity controller, a pump and the like, and accompanying processes.

Further, steam explosion using both chemical pretreatment and chemical post-treatment is also problematic in that chemicals, such as strong acid, strong alkali and the like, are used, so that corrosion occurs in the process and waste acid or waste alkali is produced, with the result that secondary environmental pollution is caused, and it is difficult to recover and regenerate media in the process.

As described above, commonly-known physical pretreatment methods are problematic in that processing is slow and energy consumption is high, thus decreasing economic efficiency, and commonly-known chemical pretreatment methods are problematic in that strong acid or strong alkali compounds are used, so that costs increase, a large-capacity process is not suitable, and toxicity becomes high, thereby having a bad influence on environment.

Therefore, it is required to develop an effective pretreatment method which does not cause environmental problems and which can reduce energy consumption.

### SUMMARY

The present inventors have continuously made research and efforts to develop a lignocellulosic biomass pretreatment process which can improve the hydrolysis efficiency of lignocellulosic biomass. As a result, they found that the surface area of lignocellulosic biomass was remarkably increased by combination of wet-milling and popping pretreatment. Based on this finding, the present invention was completed.

Accordingly, an object of the present invention is to provide a method of pretreating lignocellulosic biomass prior to hydrolysis, which can reduce energy consumption and which is environment-friendly because chemicals may not be treated, compared to conventional pretreatment methods, a method of manufacturing a sugar compound from the biomass pretreated using the method, and a method of manufacturing bioethanol from the biomass pretreated using the method.

Another object of the present invention is to provide a method of pretreating lignocellulosic biomass prior to hydrolysis, which can greatly improve the hydrolysis efficiency of lignocellulosic biomass compared to conventional pretreatment methods, a method of manufacturing a sugar compound from the biomass pretreated using the method, and a method of manufacturing bioethanol from the biomass pretreated using the method.

The objects of the present invention are not limited to the above-mentioned objects, and other objects thereof will be understandable by those skilled in the art from the following descriptions.

In order to accomplish the above objects, an aspect of the present invention provides a method of pretreating lignocellulosic biomass prior to hydrolysis, including the steps of immersing lignocellulosic biomass in water to swell the lignocellulosic biomass; wet-milling the swollen lignocellulosic biomass; and popping the wet-milled lignocellulosic biomass.

In the method, the step of popping the wet-milled lignocellulosic biomass may be performed under at least one condition of a temperature of 150˜250° C. and a pressure of 5˜25 kgf/cm² using a popping machine.

Further, the popping machine may include: a burner for directly heating a popping tank; the popping tank, which is charged with the wet-milled lignocellulosic biomass and maintains a high temperature and a high pressure when being heated by the direct heating burner; a popping product storage tank having a predetermined-shaped spatial volume and detachably connected to the popping tank such that a part of the popping tank is put into the popping product storage tank, thus recovering the lignocellulosic biomass popped in the popping tank; a motor for rotating the popping tank heated by the direct heating burner to maintain the temperature in the popping tank constant and to uniformly diffuse the steam throughout the lignocellulosic biomass charged in the popping tank; and a control unit for controlling at least one of the pressure and temperature in the popping tank.

Further, the popping tank may be provided therein with at least one of a temperature sensor and a pressure sensor.

Another aspect of the present invention provides a method of manufacturing a sugar compound from lignocellulosic biomass, including: saccharifying the lignocellulosic biomass obtained by the method of pretreating lignocellulosic biomass.

In the method, in saccharifying the lignocellulosic biomass, a saccharification enzyme may be used in an amount of 1˜20 parts by weight based on 100 parts by weight of the lignocellulosic biomass.

Further, the saccharification enzyme may be any one selected from the group consisting of cellulase, xylanase, β-glucosidase, and mixtures thereof.

Still another aspect of the present invention provides a method of manufacturing bioethanol from lignocellulosic biomass, including the steps of pretreating lignocellulosic biomass by the method of pretreating lignocellulosic biomass; saccharifying the pretreated lignocellulosic biomass to obtain a sugar compound; and fermenting the obtained sugar compound.

In the method, the saccharifying of the pretreated lignocellulosic biomass and the fermenting of the obtained sugar compound may be simultaneously performed in a single reactor.

Further, in order to simultaneously perform the saccharifying the pretreated lignocellulosic biomass and the fermenting the obtained sugar compound, a recombined strain that can perform both saccharification and fermentation may be used, and the recombined strain may be any one selected from the group consisting of Klebsiella oxytoca P2, Brettanomyces curstersii, Saccharomyces uvzrun, and Candida brassicae.

The present invention has the following advantages.

According to the method of pretreating lignocellulosic biomass prior to hydrolysis, the method of manufacturing a sugar compound from the biomass pretreated using the pretreatment method, and the method of manufacturing bioethanol from the biomass pretreated using the pretreatment method, energy consumption can be reduced, and these methods are environment-friendly because chemicals may not be treated, compared to conventional pretreatment methods.

According to the method of pretreating lignocellulosic biomass prior to hydrolysis, the method of manufacturing a sugar compound from the biomass pretreated using the pretreatment method, and the method of manufacturing bioethanol from the biomass pretreated using the pretreatment method, the hydrolysis efficiency of lignocellulosic biomass can be greatly improved, compared to conventional pretreatment methods.

### BRIEF DESCRIPTION OF DRAWINGS

FIG. 1 is a block diagram showing a process of biologically converting biomass into ethanol, the process including a pretreatment process of the present invention;

FIG. 2 is a schematic view showing a popping machine used to perform a popping process;

FIG. 3 shows electron microscope photographs showing the changes in the form of rice straw after performing a pretreatment process prior to hydrolysis according to an embodiment of the present invention; and

FIG. 4 is a graph showing the change in the hydrolysis rate of rice straw by an enzyme after performing a pretreatment process prior to hydrolysis in a method of manufacturing a sugar compound according to an embodiment of the present invention.

### DETAILED DESCRIPTION

The terms and words used in the present specification and claims should not be interpreted as being limited to typical meanings or dictionary definitions, but should be interpreted as having meanings and concepts relevant to the technical scope of the present invention based on the rule according to which an inventor can appropriately define the concept of the term to describe the best method he or she knows for carrying out the invention.

Hereinafter, exemplary embodiments of the present invention will be described in detail with reference to the accompanying drawings. However, the present invention is not limited to the following embodiments, and may be variously modified.

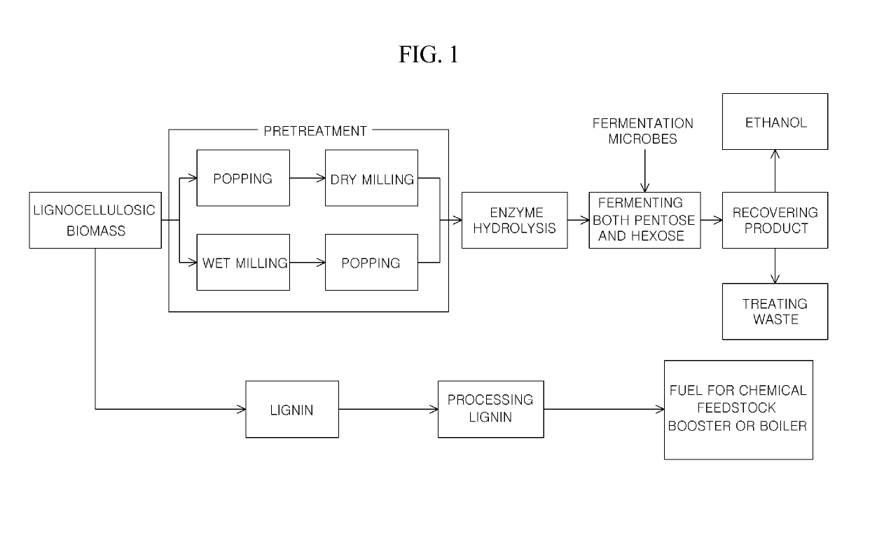

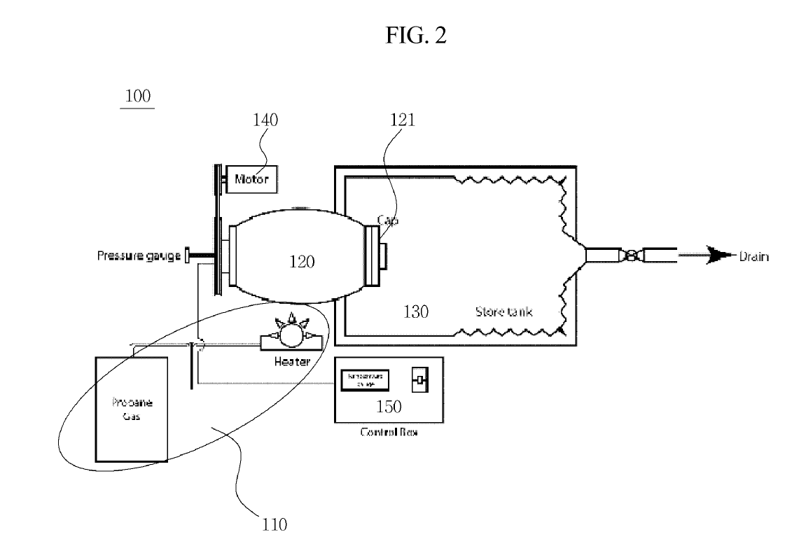

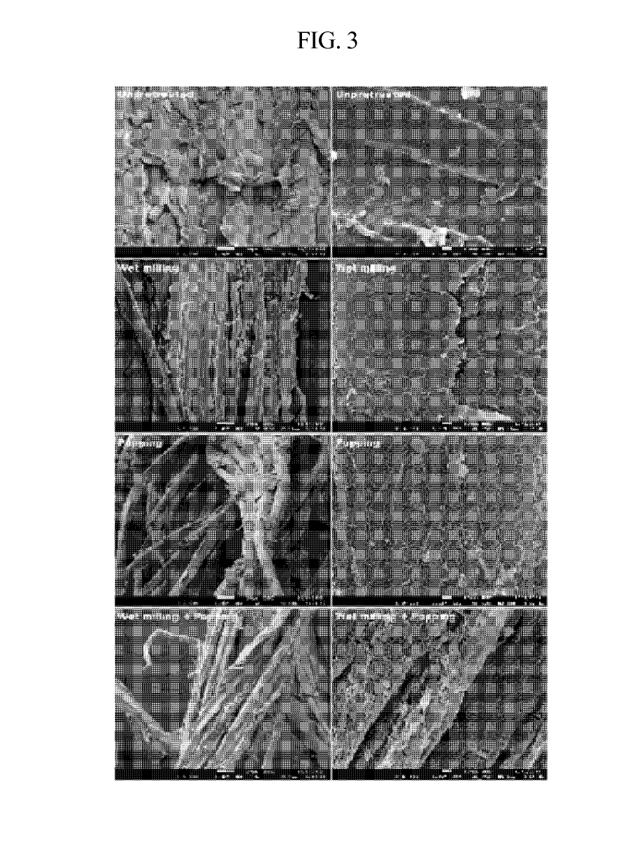

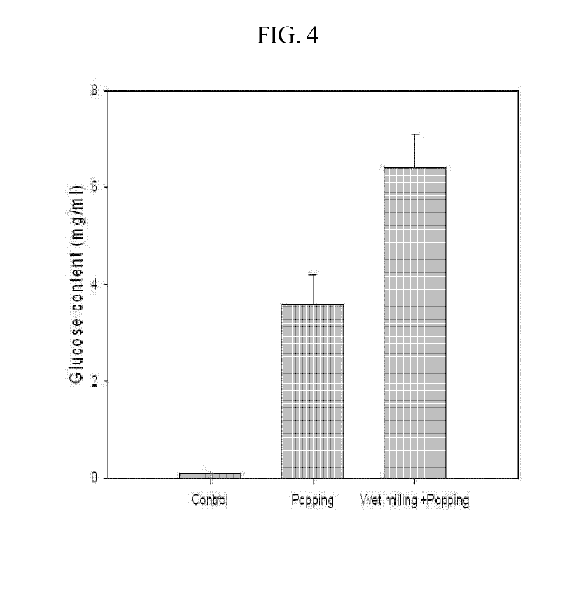

FIG. 1 is a block diagram showing a process of biologically converting biomass into ethanol, the process including a pretreatment process of the present invention; FIG. 2 is a schematic view showing a popping machine used to perform a popping process; FIG. 3 shows electron microscope photographs showing the changes in the form of rice straw after performing a pretreatment process prior to hydrolysis according to an embodiment of the present invention; and FIG. 4 is a graph showing the change in the hydrolysis rate of rice straw by an enzyme after performing a pretreatment process prior to hydrolysis in a method of manufacturing a sugar compound according to an embodiment of the present invention.

First, an embodiment of the present invention provides a method of pretreating lignocellulosic biomass prior to hydrolysis, which is required to produce a sugar compound and/or bioethanol from lignocellulosic biomass. That is, according to the method of pretreating lignocellulosic biomass, shown in FIG. 1, the surface structure of the finally-obtained pretreated lignocellulosic biomass is configured such that the contact area of the biomass with a hydrolytic enzyme is increased to remarkably increase the efficiency of hydrolyzing the biomass. Particularly, the efficiency of hydrolyzing the lignocellulosic biomass in the pretreatment process of wet-milling and then popping lignocellulosic biomass is increased by 70% or more compared to that of the pretreatment process of popping and then dry-milling lignocellulosic biomass.

Therefore, the method of pretreating lignocellulosic biomass prior to the hydrolysis according to an embodiment of the present invention includes: immersing lignocellulosic biomass in water to swell the lignocellulosic biomass; wet-milling the swollen lignocellulosic biomass; and popping the wet-milled lignocellulosic biomass. Here, the popping of the wet-milled lignocellulosic biomass may be performed at a temperature of 150˜250° C. and/or a pressure of 5˜25 kgf/cm² using a popping machine, and for example, may be performed at a temperature of 170˜250° C. and/or a pressure of 15˜25 kgf/cm².

As shown in FIG. 2, the popping machine 100, which is developed to perform a popping process, includes a direct heating burner 110, a popping tank 120, a popping product storage tank 130, a motor 140, and a control unit 150.

First, the direct heating burner 110 is an alternative to a steam generator used in steam explosion. That is, a conventional steam explosion process is performed in an indirect heating manner in which a steam generator is connected to an explosion tank by a steam jacket to maintain the inside of the explosion tank at high temperature and high pressure. In contrast, the direct heating burner 110 constituting the popping machine 100 of an embodiment of the present invention is configured such that the popping tank 120 is directly heated to maintain the inside of the popping tank 120 at high temperature and high pressure. Therefore, it can be seen that the direct heating burner 110 is very excellent in terms of heat utilization and stability compared to the steam generator used in the steam explosion process.

The popping tank 120, which is a container charged with the wet-milled biomass, may be made of a material that can resist high temperature and high pressure and can be directly fired because it must be stable under high temperature and high pressure. One side of the popping tank 120 is fixed on a well-known frame such that the popping tank 120 can be rotated by a motor 140, and the other side thereof is provided such that the opening 121 for charging or discharging a sample (wet-milled biomass) can be sealed with a cap. In this case, the opening 121 may be provided with a hatch such that the steam contained in the sample after popping is instantaneously discharged.

The popping tank 120 is configured such that a temperature sensor (not shown) for detecting the temperature of the inside of the popping tank 120 is provided in the popping tank 120, to transfer the detected temperature to the control unit 150. If necessary, the popping tank 120 may be configured such that a pressure sensor is mounted inside the popping tank 120 instead of mounting a pressure gauge outside the popping tank 120.

The popping product storage tank 130 has a predetermined-shaped spatial volume and serves to recover the popped sample. As shown in FIG. 2, the popping product storage tank 130 may be detachably connected to the popping tank 120 such that a part of the popping tank 120 is put into the popping product storage tank 130. Further, as shown in FIG. 2, the popping product storage tank 130 may be provided with an outlet for discharging the popped biomass to the side opposite to the side to which the popping tank 120 is connected.

The motor 140 serves to rotate the popping tank 120 to allow the temperature in the popping tank 120 and the diffusion of steam in the sample to be uniform when the temperature in the popping tank 120 is increased by the direct heating burner 110.

The control unit 150 is provided in the form of a control box including a keypad and a display window, and can control pressure and temperature by closing a valve between heaters of the direct heating burner 110 at the set pressure and/or temperature as well as control the motor 140.

In detail, the process of saccharifying the pretreated biomass may generally be performed by acid saccharification. However, in an embodiment of the present invention, the process of saccharifying the pretreated biomass may be performed by enzymatic saccharification without the addition of any chemical. The saccharification enzyme used in the enzymatic saccharification may be selected from the group consisting of cellulase, xylanase, β-glucosidase, and mixtures thereof. For example, the saccharification enzyme may be a mixture of cellulase and xylanase having a weight ratio of 1˜2:1˜2, particularly, 2:1. The saccharification enzyme may be used in an amount of 1˜20 parts by weight based on 100 parts by weight of biomass. The process of saccharifying the pretreated biomass may be performed at a temperature of 40˜45° C. for 6˜24 hours, particularly, 24 hours.

Further, in an embodiment of the present invention, yeast, for example, Saccharomyces cerevisiae may be used as a fermentation strain for producing bioethanol. The fermentation strain may be any one selected from sugar-resistant strains that can perform fermentation even at high sugar concentration; heat-resistant strains that can convert biomass into ethanol even at a temperature of 40˜45° C. which is the optimum saccharification temperature; and recombined strains that can perform both saccharification and fermentation, such as Klebsiella oxytoca P2, Brettanomyces curstersii, Saccharomyces uvzrun, Candida brassicae, and the like, which are well known to those skilled in the art. The fermentation process may be performed at a temperature of 25˜30° C., particularly, 30° C. for 12˜24 hours, independently with the saccharification process. The fermentation process and the saccharification process may be simultaneously performed.

### Example 1

100 g of rice straw harvested in the autumn was immersed in water for one day to sufficiently swell the rice straw. Thereafter, the swollen rice straw and water were wet-milled using a milling machine to obtain a finely wet-milled product. Subsequently, the wet-milled product was put into a popping machine shown in FIG. 2, and was then popped at a temperature of 200° C. and a pressure of 21 kgf/cm² to obtain a pretreated rice straw product (wet milling+popping) of the present invention.

### Comparative Example 1

100 mg of rice straw harvested in the autumn was sufficiently immersed in water for one day to swell the rice straw. Thereafter, the swollen rice straw was put into a popping machine shown in FIG. 2, was popped at a temperature of 200° C. and a pressure of 21 kgf/cm², and was then pulverized to obtain a comparative pretreated rice straw product (popping).

### Comparative Example 2

100 mg of rice straw harvested in the autumn was sufficiently immersed in water for one day to swell the rice straw. Thereafter, the swollen rice straw and water were wet-milled using a milling machine to obtain a comparative finely wet-milled pretreated product (control).

### Experimental Example 1

The pretreated products obtained from Example 1 and Comparative Examples 1 and 2 were observed by an electron microscope, and the photographs thereof are shown in FIG. 2. Further, the chemical components of the pretreated products were analyzed, and the results thereof are given in Table 1 below.

TABLE 1

| | Rahm- | Arabi- | Man- | Galac- | Xy- | Glu- | |
|---|---|---|---|---|---|---|---|
| | nose | nose | nose | tose | lose | cose | Total |
| Control | 0.1 | 2.9 | 0.6 | 0.7 | 17.9 | 48.2 | 70.4 |
| Comparative | 0.1 | 2.7 | 0.5 | 1.4 | 16.3 | 46.8 | 67.8 |
| Example 1 | | | | | | | |
| (popping) | | | | | | | |
| Example 1 | 0.4 | 2.9 | 0.8 | 1.1 | 18.1 | 45.0 | 68.2 |
| (wet milling + | | | | | | | |
| popping) | | | | | | | |

As given in Table 1 above, it can be seen that the change of the chemical components of the pretreated product (wet milling+popping) obtained by wet-milling and then popping rice straw, the change of the pretreated product (popping) obtained by popping and then pulverizing rice straw and the change of the pretreated product (control) obtained by wet-milling rice straw were not greatly different from each other. However, as shown in FIG. 3, it can be seen that the physical and morphological changes thereof observed using an electron microscope were greatly different from each other.

Further, as shown in FIG. 3, it can be seen that the surface area of the pretreated product (control) obtained by immersing and then wet-milling rice straw is larger than that of the non-pretreated product, and the surface area of the pretreated product (popping) obtained by immersing and then popping rice straw is larger than that of the pretreated product (control) obtained by immersing and then wet-milling rice straw. In particular, it can be seen that there is a remarkable increase in the surface area of the pretreated product (wet milling+popping) obtained by immersing, wet-milling and then popping rice straw.

### Example 2

Cellulase (600 U/g biomass) and xylanase (300 U/g biomass) were added to 50 mg of the pretreated rice straw product (wet milling+popping) obtained from Example 1, and then this pretreated rice straw product was saccharified at a temperature of 37° C. for 24 hours to obtain a sugar compound (wet milling+popping).

### Comparative Example 3

Cellulase (600 U/g biomass) and xylanase (300 U/g biomass) were added to 50 mg of each of the pretreated rice straw products obtained from Comparative Examples 1 and 2, and then these pretreated rice straw products were respectively saccharified at a temperature of 37° C. for 24 hours to obtain a comparative sugar compound (popping) and a comparative sugar compound (control).

### Experimental Example 2

The concentrations of the comparative sugar compound (popping) and comparative sugar compound (control) obtained from Comparative Example 3 and the sugar compound (wet milling+popping) obtained from Example 2 were measured using high performance liquid chromatography (HPLC), and the results thereof are shown in FIG. 4.

From FIG. 4, it can be seen that the comparative sugar compound (control) has a low hydrolysis rate of 0.1 mg/ml, and the comparative sugar compound (popping) has a relatively high hydrolysis rate of 3.6 mg/ml, but that the sugar compound (wet milling+popping) obtained from Example 2 has a high hydrolysis rate of 6.4 mg/ml. Therefore, it can be seen that the hydrolysis rate of the sugar compound (wet milling+popping) is 64 times greater than that of the comparative sugar compound (control), and is about twice (70% or more) greater than that of the comparative sugar compound (popping).

From the above results, it can be seen that, among the pretreatment methods of the embodiment of the present invention, the composite pretreatment method of immersing, wet-milling and then popping biomass is excellent compared to the pretreatment method of immersing, popping and then pulverizing biomass. Therefore, it can be clearly understood that, among the pretreatment methods of the present invention, the composite pretreatment method of immersing, wet-milling and then popping biomass is suitable for a biotechnological process using an enzyme.

### Example 3

### Production of Bioethanol

Pretreatment Process Prior to Hydrolysis

100 g of rice straw harvested in the autumn was immersed in water for one day to sufficiently swell the rice straw. Thereafter, the swollen rice straw and water were wet-milled using a milling machine to obtain a finely wet-milled product. Subsequently, the wet-milled product was put into a popping machine shown in FIG. 2, and was then popped at a temperature of 200° C. and a pressure of 21 kgf/cm² to obtain a pretreated rice straw product.

2. Saccharification Process

Cellulase (600 U/g biomass) and xylanase (300 U/g biomass) were added to 50 mg of the pretreated rice straw product, and then this pretreated rice straw product was saccharified at a temperature of 37° C. for 24 hours to obtain a sugar compound, that is, glucose.

3. Fermentation Process

The obtained glucose (sugar compound) was concentrated to a concentration of 10%. Subsequently, 15 g/l of the concentrated glucose was added to Saccharomyces cerevisiae (fermentation strain for producing bioethanol), and was then fermented at a temperature of 30° C. for 24 hours to obtain bioethanol.

In this case, the saccharification process and the fermentation process may be performed at the same time.

In the above Examples, rice straw harvested in the autumn was used as lignocellulose biomass. However, the scope of the present invention is not limited thereto because the rice straw can be used in the present invention as long as it contains lignocellulose biomass.

Although the exemplary embodiments of the present invention have been disclosed for illustrative purposes, those skilled in the art will appreciate that various modifications, additions and substitutions are possible, without departing from the scope and spirit of the invention as disclosed in the accompanying claims.

1. A method of pretreating lignocellulosic biomass prior to hydrolysis, comprising:
immersing lignocellulosic biomass in water to swell the lignocellulosic biomass;
wet-milling the swelled lignocellulosic biomass; and
popping the wet-milled lignocellulosic biomass.

2. The method of claim 1, wherein the popping of the wet-milled lignocellulosic biomass is performed under at least one condition of a temperature of 150˜250° C. and a pressure of 5˜25 kgf/cm² using a popping machine.

3. The method of claim 2, wherein the popping machine comprises:
a burner for directly heating a popping tank;
the popping tank, which is charged with the wet-milled lignocellulosic biomass and maintains a high temperature and a high pressure when being heated by the direct heating burner;
a popping product storage tank having a predetermined-shaped spatial volume and detachably connected to the popping tank such that a part of the popping tank is put into the popping product storage tank, thus recovering the lignocellulosic biomass popped in the popping tank;
a motor for rotating the popping tank heated by the direct heating burner to maintain the temperature in the popping tank constant and to uniformly diffuse the steam throughout the lignocellulosic biomass charged in the popping tank; and
a control unit for controlling at least one of the pressure and temperature in the popping tank.

4. The method of claim 3, wherein the popping tank is provided therein with at least one of a temperature sensor and a pressure sensor.

5. A method of manufacturing a sugar compound from lignocellulosic biomass, comprising:
immersing lignocellulosic biomass in water to swell the lignocellulosic biomass;
wet-milling the swelled lignocellulosic biomass;
popping the wet-milled lignocellulosic biomass; and
saccharifying the popped biomass.

6. The method of claim 5, wherein, in the saccharifying of the lignocellulosic biomass, a saccharification enzyme is used in an amount of 1˜20 parts by weight based on 100 parts by weight of the lignocellulosic biomass.

7. The method of claim 6, wherein the saccharification enzyme is selected from the group consisting of cellulase, xylanase, β-glucosidase, and a combination thereof.

8. A method of manufacturing bioethanol from lignocellulosic biomass, comprising:
pretreating lignocellulosic biomass by immersing lignocellulosic biomass in water to swell the lignocellulosic biomass, wet-milling the swelled lignocellulosic biomass, and popping the wet-milled lignocellulosic biomass;
saccharifying the pretreated lignocellulosic biomass to obtain a sugar compound; and
fermenting the obtained sugar compound.

9. The method of claim 8, wherein the saccharifying of the pretreated lignocellulosic biomass and the fermenting of the obtained sugar compound are simultaneously performed in a single reactor.

10. The method of claim 9, wherein, in order to simultaneously perform the saccharifying and the fermenting, a recombined strain that can perform both saccharification and fermentation is used, and the recombined strain is selected from the group consisting of Klebsiella oxytoca P2, Brettanomyces curstersii, Saccharomyces uvzrun, Candida brassicae, and a combination thereof.

11. The method of claim 5, wherein the popping of the wet-milled lignocellulosic biomass is performed under at least one condition of a temperature of 150˜250° C. and a pressure of 5˜25 kgf/cm² using a popping machine.

12. The method of claim 11, wherein the popping machine comprises:
a burner for directly heating a popping tank;
the popping tank, which is charged with the wet-milled lignocellulosic biomass and maintains a high temperature and a high pressure when being heated by the direct heating burner;
a popping product storage tank having a predetermined-shaped spatial volume and detachably connected to the popping tank such that a part of the popping tank is put into the popping product storage tank, thus recovering the lignocellulosic biomass popped in the popping tank;
a motor for rotating the popping tank heated by the direct heating burner to maintain the temperature in the popping tank constant and to uniformly diffuse the steam throughout the lignocellulosic biomass charged in the popping tank; and
a control unit for controlling at least one of the pressure and temperature in the popping tank.

13. The method of claim 12, wherein the popping tank is provided therein with at least one of a temperature sensor and a pressure sensor.

14. The method of claim 8, wherein the popping of the wet-milled lignocellulosic biomass is performed under at least one condition of a temperature of 150˜250° C. and a pressure of 5˜25 kgf/cm² using a popping machine.

15. The method of claim 14, wherein the popping machine comprises:
a burner for directly heating a popping tank;
the popping tank, which is charged with the wet-milled lignocellulosic biomass and maintains a high temperature and a high pressure when being heated by the direct heating burner;
a popping product storage tank having a predetermined-shaped spatial volume and detachably connected to the popping tank such that a part of the popping tank is put into the popping product storage tank, thus recovering the lignocellulosic biomass popped in the popping tank;
a motor for rotating the popping tank heated by the direct heating burner to maintain the temperature in the popping tank constant and to uniformly diffuse the steam throughout the lignocellulosic biomass charged in the popping tank; and
a control unit for controlling at least one of the pressure and temperature in the popping tank.

16. The method of claim 15, wherein the popping tank is provided therein with at least one of a temperature sensor and a pressure sensor.
